(19) United States
(12) Plant Patent Application Publication
(10) Pub. No.: US 2015/0143597 P1
(43) Pub. Date: May 21, 2015

(54) Pelargonium plant named 'KLEIZ13338'

(50) ;

(71) Applicant: Klemm+Sohn GmbH & Co. KG, Stuttgart (DE)

(72) Inventors: Nils Klemm, Stuttgart (DE); Martin Glawe, Stuttgart (DE)

(73) Assignee: Klemm+Sohn GmbH & Co. KG, Stuttgart (DE)

(21) Appl. No.: 13/998,657

(22) Filed: Nov. 20, 2013

(51) Int. Cl. A01H 5/00

(52) U.S. Cl. PLT/324

(57) ABSTRACT

A new Pelargonium plant particularly distinguished by having semi-double flowers with a white color, compact growth habit and strong branching, is disclosed.

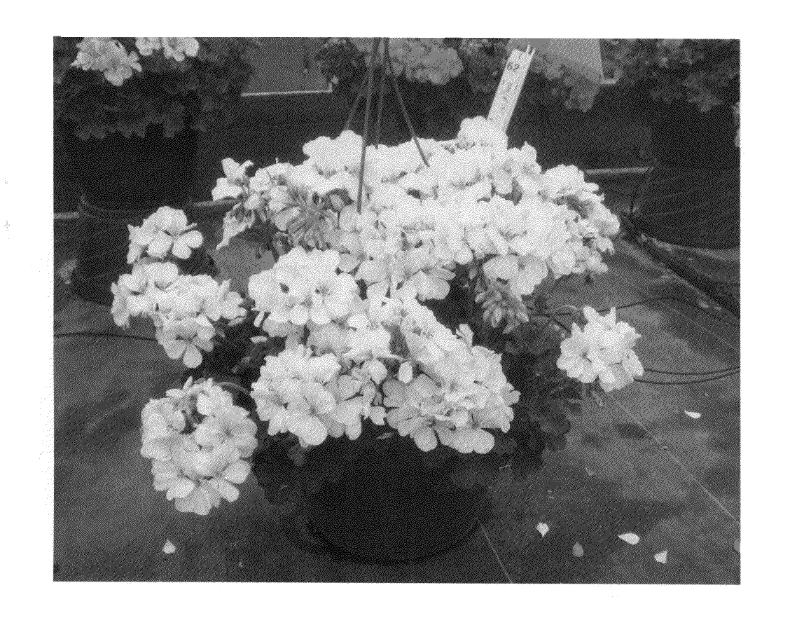

### GENUS AND SPECIES

Pelargonium sp.

### VARIETY DENOMINATION

‘KLEIZ13338’

### BACKGROUND OF THE NEW PLANT

The present invention comprises a new and distinct variety of Pelargonium, botanically known as Pelargonium sp. and hereinafter referred to by the variety name ‘KLEIZ13338’. ‘KLEIZ13338’ originated from a controlled cross between the female Pelargonium variety ‘Cassandra’ (U.S. Plant Pat. No. 6,220), having white flowers with medium branching and a strong growth habit, and the proprietary male Pelargonium variety ‘PP 06 086’ (unpatented), having orange flowers, with small branching and a small growth habit.

In June 2008 the two Pelargonium parental lines ‘Cassandra’ and ‘PP 06 086’ were crossed in Stuttgart, Germany and seeds were obtained. The seeds were sown and plants were grown for evaluation. A plant line was selected in June 2009 and named ‘KLEIZ13338’. In June 2009 ‘KLEIZ13338’ was first vegetatively propagated by vegetative cuttings. ‘KLEIZ13338’ was found to reproduce true to type in successive generations of asexual propagation via vegetative cuttings in Stuttgart, Germany.

### CROSS REFERENCE TO RELATED APPLICATIONS

Plant Breeder's Rights for ‘KLEIZ13338’ were applied for in Canada on Feb. 8, 2013, Application No. 13-7923. ‘KLEIZ13338’ has not been made publicly available or sold more than one year prior to the filing date of this application.

### SUMMARY

The following are the most outstanding and distinguishing characteristics of this new variety when grown under normal horticultural practices in Stuttgart, Germany.

1. White flower color;

2. Semi-double flowers;

3. Compact growth habit; and

4. Strong branching.

### DESCRIPTION OF THE PHOTOGRAPH

This new Pelargonium plant is illustrated by the accompanying photograph which shows an overall view of the plant. The photograph is of a 4 to 5 month-old plant grown in Stuttgart, Germany under greenhouse conditions in May 2012. The colors shown are as true as can be reasonably obtained by conventional photographic procedures.

### DESCRIPTION OF THE NEW VARIETY

The following detailed descriptions set forth the distinctive characteristics of ‘KLEIZ13338’. The data which define these characteristics were collected from asexual reproductions carried out in Stuttgart, Germany. Data was collected on fifteen-week-old plants grown in an indoor trial in Stuttgart, Germany under greenhouse conditions in April 2013. Color references are to The R.H.S. Colour Chart of The Royal Horticultural Society of London (R.H.S.), 5^{th} edition (2007).
Classification:
   Family.—Geraniaceae.
   Botanical.—Pelargonium sp.
   Common.—Pelargonium.
   Designation.—‘KLEIZ13338’.
Parentage:
   Female parent.—Pelargonium line ‘Cassandra’ (U.S. Plant Pat. No. 6,220).
   Male parent.—Pelargonium line ‘PP 06 086’ (unpatented).
Plant:
   Habit.—Upright.
   Height.—20.0 cm.
   Spread.—20.0 cm.
   Life cycle.—Annual, January to April in a greenhouse, May to October outside.
   Form.—Rounded, potted plant.
   Time to produce a rooted cutting.—18 days.
   Time to bloom from propagation.—75 days.
   Lastingness of inflorescence on the plant.—About 2 weeks.
Stems:
   Color.—RHS 137C.
   Pubescence.—Present.
   Stem form.—Round.
   Diameter.—0.6 cm.
   Length.—8.0 cm to 12.0 cm.
   Internode length.—1.5 cm to 2.0 cm.
   Anthocyanin.—Absent.
Leaves:
   Arrangement.—Alternate.
   Shape.—Round, ivy shaped.
   Apex.—Obtuse.
   Base.—Open.
   Margin.—Crenate.
   Surface Texture.—Upper surface: Rough Lower surface: Smooth.
   Length.—5.0 cm.
   Width.—6.0 cm.
   Color.—Upper surface: RHS 137A Lower surface: RHS 137D.
   Surface pubescence.—Upper surface: Sparse Lower surface: Absent.
   Petiole.—Length: 5.0 cm to 7.0 cm Color: RHS 137C.
   Venation pattern.—Palmate.
   Venation color.—Upper surface: RHS 138A Lower surface: RHS 138B.
Inflorescence:
   Umbels.—Length: 5.0 cm to 6.0 cm Diameter: 8.0 cm.
   Individual florets of the umbel.—Depth 1.0 cm Diameter: 3.5 cm.
   Fragrance.—Absent.
Flower buds:
   Cluster shape.—Round.
   Cluster size.—Length: 2.0 cm Width: 2.0 cm.
   Texture.—Rough.
   Bud.—Shape: Elliptic, ovate Apex: Cuspidate Base: Obtuse Margin: Smooth Color: RHS 138A.
Petals:
   Quantity per flower.—5.
   Shape.—Ovate.
   Apex.—Smooth, round.
   Base.—Obtuse.
   Margin.—Entire.
   Length.—2.0 cm.
   Width.—1.0 cm.
   Color of lobes.—Upper and lower lobes: Upper surface: RHS 157B Lower surface: RHS 157C.
Sepals:
   Number per flower.—5.
   Shape.—Elliptic to ovate.
   Apex.—Acute.
   Base.—Obtuse.
   Margin.—Entire.
   Color.—Upper surface: RHS 138B Lower surface: RHS 138D.
   Length.—0.9 cm.
   Width.—0.4 cm.
Pedicel:
   Length.—0.5 cm to 1.2 cm.
   Diameter.—0.15 cm.
   Color.—RHS 138D.
Peduncle:
   Length.—10.0 cm to 15.0 cm.
   Diameter.—0.5 cm.
   Color.—RHS 137D.
   Texture.—Rough.
Reproductive organs:
   Stamens.—Anther length: 0.2 cm Filament length: 0.6 cm Filament color: RHS N155B Pollen amount: Moderate Pollen color: RHS 25D.
   Pistils.—Number: 1 Stigma color: RHS 68C Style length: 0.1 cm Style color: RHS 56D Ovary arrangement: Erect.
Fruit and seed set: None observed
Disease and pest/insect resistance: None observed

### COMPARISON WITH PARENTAL LINES AND KNOWN VARIETY

‘KLEIZ13338’ is distinguished from its parents as described in Table 1:

TABLE 1
Comparison with Parental Lines

| | | Female parent | Male Parent |
|---|---|---|---|
| Trait | ‘KLEIZ13338’ | ‘Cassandra’ | ‘PP 06 086’ |
| Growth habit | Compact | Strong | Small |
| Flower color | White | White | Orange |
| Branching | Strong | Medium | Small, little |

‘KLEIZ13338’ is most similar to the commercial Pelargonium plant named ‘Cassandra’ (U.S. Plant Pat. No. 6,220). Differences between the two varieties are described in Table 2:

TABLE 2
Comparison with Similar Variety

| | | Commercial line |
|---|---|---|
| Trait | ‘KLEIZ13338’ | ‘Cassandra’ |
| Flower fullness | Semi-double flowers | Double flowers |
| Growth habit | Compact | Strong |

We claim:

1. A new and distinct variety of Pelargonium plant named ‘KLEIZ13338’ as shown and described herein.
